(19) United States
(12) Patent Application Publication
(10) Pub. No.: US 2009/0111096 A1
(43) Pub. Date: Apr. 30, 2009

(54) Method of exhaustive analysis of transcriptionally-active domain (non-methylated domain) on genome

(75) Inventor: Masumi Abe, Chiba-shi (JP)

Correspondence Address: BIRCH STEWART KOLASCH & BIRCH, PO BOX 747, FALLS CHURCH, VA, 22040-0747, US

(73) Assignee: National Institute of Radiological Science, Chiba (JP)

(21) Appl. No.: 11/664,877

(22) PCT Filed: Sep. 6, 2005
(86) PCT No.: PCT/JP2005/016344

§ 371 Date: Oct. 2, 2007

(30) Foreign Application Priority Data
Oct. 6, 2004 (JP) 2004-293671

(51) Int. Cl. C12Q 1/68

(52) U.S. Cl. 435/6

(57) ABSTRACT

The purpose of the present invention is to provide a method to simultaneously detect a number of the non-methylated regions in the genome of two or more types of cells, and to exhaustively compare and analyze them.

The present invention relates to a method for the detection of a non-methylated region in genome comprising preparing the DNA fragment population consisting only of DNA fragments derived from the non-methylated regions with use of a methylation-sensitive enzyme for at least either of the first restriction enzyme X and the second restriction enzyme Y, and detecting the non-methylated region in genome utilizing the principle of HiCEP, and to a method for analyzing the change in a transcriptionally-active region in genome comprising detecting a non-methylated region in genome derived from more than two kinds of cells by the above method, comparing the results such as, for example, change of a peak area corresponding to an amount of the non-methylated region to analyze difference in the non-methylated region.

### FIELD OF THE INVENTION

The invention relates to a method for analysis of genomic diversity, more particularly to a method of exhaustive analysis or detection of transcriptionally-active region (non-methylated region) in genome.

### BACKGROUND OF THE INVENTION

Now that the genome sequences of human and mouse have been completely or almost completely determined, the focus of post-genome analysis has now shifted into difference in genomic base sequences between individuals or diseases, and their causal relationship, i.e., any differences in the base sequences between a healthy subject and a patient, and any relationship between these differences and diseases.

For example, there is known a method of exhaustively identifying SNP(single nucleotide polymorphism) or microsatellite (a repeated sequence scattered in the genome, being different in the number of the repeated sequences among individuals) present in the genomic base sequences and comparing them between individuals or diseases (or between the healthy subject and the patient) in order to find any relationship in the researches using the SNP or microsatellite as a polymorphism marker.

Some of the relationship between the polymorphism marker and disease have been revealed by the above researches. However, since the polymorphism marker only reflects the differences in the genomic base sequences, whether such difference may be directly associated with diseases or not is still left to further researches in the future. In order to analyze the above relationship more in detail, it would be necessary to analyze the way of expression of disease-related genes (not only those causing the diseases due to their defects, but also those directly and indirectly involved in the diseases), or to exhaustively analyze expression frequency of the above genes.

On the other hand, modification of a base in the genome is known as a factor involved in transcription-expression mechanism in addition to the above differences in the base sequences. The most well-known factor is methylation control of a cytidine base in CG base sequence. Thus, if the cytidine residue is methylated (Met-C) at a site which is involved in a transcriptionally-regulating sequence or a transcripted sequence, the transcription of a gene involved therein will be inhibited or controlled (Non Patent Document 1). It is said that about 60-90% of the CG sequences in the genome are methylated in a certain cell condition, and such methylation take a role in the control of gene-transcription (Non Patent Document 2).

Up to now, there have been reported methods of analysis of methylation of particular sites in the genome, such as an analytical method comprising cleaving a particular part of the genome with a methylation-sensitive restriction enzyme and a methylation-insensitive restriction enzyme, amplifying the cleaved fragment followed by analysis with electrophoresis and the like (Non Patent Document 3), and a method of analysis of a methylation site using an adaptor specific to a methylation-insensitive restriction enzyme, XmaI, that will produce an cohesive end with (Non Patent Document 4).

Furthermore, Non Patent Document 1 describes a method for the detection of a methylated site in the genome, and a bio-chip used in the method.

[Non Patent Document 1] Tatsuya K., Akio A., Experimental Medicine, Extra Number Vol. 021, 1442-1447, 2003

[Non Patent Document 2] Razin A, Riggs A D., Science, 1980 Nov. 7: 210 (4470): 604-10

[Non Patent Document 3] Toshikazu U., et al., Proc. Natal. Acad. Sci., USA Vol. 94, pp. 2284-2289, March 1997

[Non Patent Document 4] Minoru T., et al., Cancer Research 59, 2307-2312 (1999)

[Patent Document 1] Japanese Patent Application Publication 2003-38183

### DISCLOSURE OF THE INVENTION

### Problems to be Solved by the Invention

As already mentioned above, it will be necessary to carry out correlated analysis between the differences in the genomic base sequences and the transcriptionally-active region in the genome in order to identify the disease-related genes. Thus, the purpose of the present invention is to provide a method to simultaneously detect a number of the non-methylated regions in the genome of two or more types of cells, and to exhaustively compare and analyze them.

### Means for Solving the Problems

A first aspect of the present invention relates to a method for the detection of a non-methylated region in genome comprising:
(a) a step of cleaving a genomic DNA with a first restriction enzyme X;
(b) a step of binding an X adaptor comprising a complementary sequence to a cleaved site by the first restriction enzyme X, a complementary sequence to an X primer and a tag substance added to the other end of the complementary sequence to the cleaved site, to the cleaved site of the DNA fragment cleaved by the first restriction enzyme X in the step (a) so as to prepare a DNA fragment having the X adaptor bound to the cleaved site;
(c) a step of cleaving the DNA fragment having the X adaptor bound thereto and prepared in the step (b) with a second restriction enzyme Y that does not cleave a sequence complementary to the X primer;
(d) a step of separating and purifying the DNA fragment having the X adaptor bound thereto and cleaved in the step (c) with a substance having a high affinity for the tag substance added to the X adaptor;
(e) a step of binding a Y adaptor comprising a complementary sequence to the cleaved site by the second restriction enzyme Y and a complementary sequence to a Y primer, to the cleaved site by the second restriction enzyme Y of the DNA fragment having the X adaptor bound thereto and purified in the step (d) so as to prepare a DNA fragment having the X adaptor and Y adaptor bound to its both ends;
(g) a step of PCR with the use of a double-stranded DNA sequence prepared in the step (e) as a template and a primer set of X1 primer comprising a two-base sequence of N₁N₂ (N₁ and N₂ are a base selected from the group consisting of adenine, thymine, guanine and cytosine, being the same or different with each other) at its 3′ end and Y1 primer comprising a two-base sequence of N₃N₄ (N₃ and N₄ are a base selected from the group consisting of adenine, thymine, guanine and cytosine, being the same or different with each other) at its 31 end; and
(h) a step of separating and detecting the resulting PCR products in accordance with their chain length;
   wherein either of the first restriction enzyme X and the second restriction enzyme Y is a methylation-sensitive enzyme.

A second aspect of the present invention relates to a method for analyzing the change in a transcriptionally-active region in genome comprising detecting a non-methylated region in genome derived from more than two kinds of cells by the above method, comparing the results such as, for example, change of a peak area corresponding to an amount of the non-methylated region to analyze difference in the non-methylated region. The genome may be derived from any sources such as eucaryotic cells, especially genome from mammalian cells including human and mouse cells. The phrase “more than two kinds of cells” widely means that a certain feature of the cells is different with each other depending on the kinds of species, organ, tissue, the stage of generation or differentiation, and condition of diseases.

### ADVANTAGES OF THE INVENTION

In the method for the detection of the non-methylated region in genome, as at least one of the first restriction enzyme X and the second restriction enzyme Y is a methylation-sensitive enzyme that can not cleave when a cytidine modified with methylation is present, it will be possible to carry out fragmentation of genome regions comprising the methylated region or non-methylated region and to exhaustively separate and detect the resulting fragment population (fragment library) with a high sensitivity.

Furthermore, by comparing the results obtained in the present analyzing method with respect to more than two kinds of cells that have any difference, such as those derived from a patient and a healthy individual, or those derived from different stages in generation or differentiation, it will be easy to detect and analyze the non-methylated region, i.e., transcriptionally-active region.

### BRIEF DESCRIPTION OF DRAWINGS

FIG. 1 shows the results of the step (h) when the combination of “X-AA” and “Y-AA” is used in the step (g).

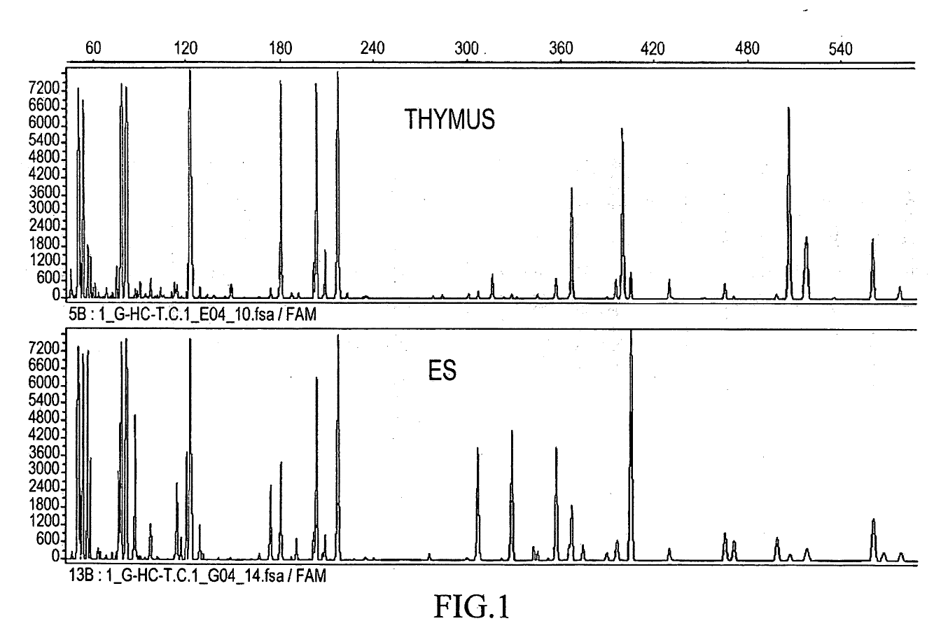

FIG. 2 shows the results of the step (h) when the combination of “X-CA” and “Y-AC” is used in the step (g).

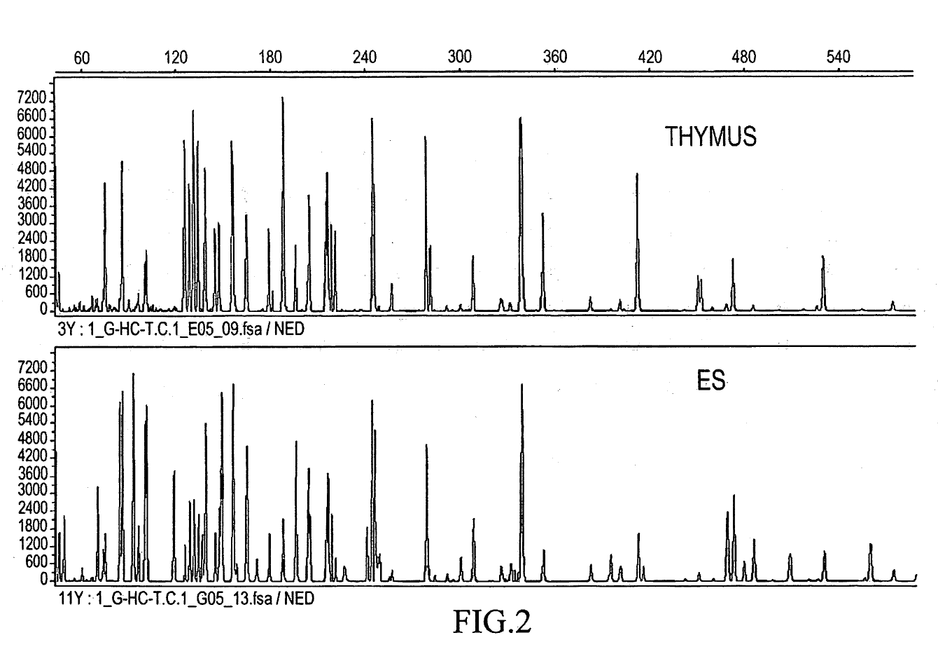

FIG. 3 shows the results of the step (h) when the combination of “X-GG” and “Y-CC” is used in the step (g).

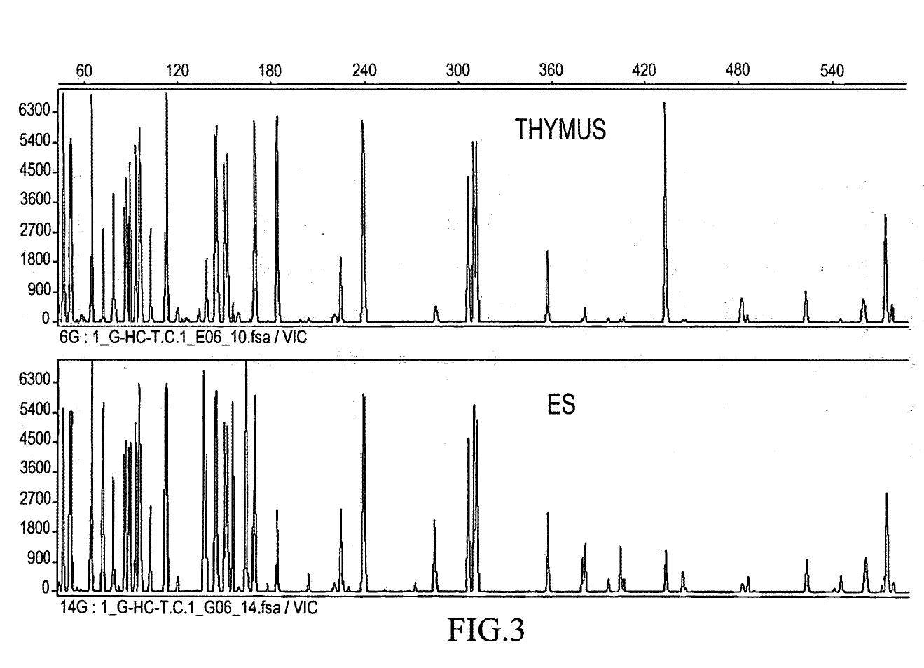

### BEST MODE FOR CARRYING OUT THE INVENTION

In order to obtain a satisfactory detection sensitivity even if a sufficient amount of a starting genomic DNA is not available, it is preferable to insert between the steps (e) and (g) a step (f) of amplifying the DNA fragment having the X adaptor and Y adaptor bound to its both ends and prepared in the step (e) by means of PCR with the use of said DNA fragment as a template and a primer set of the X primer and Y primer. As a result, the number of the double-stranded DNA supplemented with the X primer and Y primer can be amplified. Those skilled in the art may amplify the DNA fragment 128-1624 times by optionally selecting the conditions such as, for example, by setting the number of PCR cycles between 7 and 10.

In the method for the detection according to the present invention, a step (i) of identifying the detected peak may be comprised after separating and detecting the resulting PCR products. Such identification may be done in any manner known for those skilled in the art. For example, the detected peak is collected so that their base sequences may be specifically determined by an appropriate experimental manner such as a suitable sequencing method. Alternatively, those sequences may be theoretically determined my means of a computer system. For example, by utilizing data available from any data base known for those skilled in the art, such as GenBank, EMBL and DDBJ, a DNA fragment that is expected to be cleaved by the restriction enzymes used in the present invention may be theoretically predicted. Accordingly, comparison of such predicted data with data actually determined by the present method can identify which gene in the genome a DNA fragment in concern is derived from.

In the present method, it is necessary that either of the first restriction enzyme X and the second restriction enzyme Y should be a methylation-sensitive enzyme in order to detect the non-methylated region in the genome.

Specifically, in preferred aspects, the first restriction enzyme X is a methylation-sensitive enzyme, and the second restriction enzyme Y is a methylation-sensitive enzyme or methylation-insensitive enzyme. Alternatively, the first restriction enzyme X is a methylation-insensitive enzyme, and the second restriction enzyme Y is a methylation-sensitive enzyme. Any enzyme known for those skilled in the art may be used as the first restriction enzyme X and second restriction enzyme Y.

In order to make it easier to identify a location in the genome, as the first restriction enzyme X are preferred those having a low frequency of appearance such as 6-base recognizing methylation-sensitive enzymes including SalI (manufactured by Takara Bio. Inc.: recognition sequence:GTCGAC) and BssHII (manufactured by Takara Bio. Inc.: recognition sequence:GCGCGC), and 8-base recognizing methylation-sensitive enzymes including NotI (manufactured by Takara Bio. Inc.: recognition sequence:GCGGCCGC) and AscI (manufactured by New England BioLabs: recognition sequence:GGCGCGCC).

Similarly, as the first restriction enzyme X are preferred the methylation-insensitive enzymes having a low frequency of appearance such as Xmal (manufactured by New England BioLabs: recognition sequence:CCCGGG), BssSI (manufactured by New England BioLabs: recognition sequence:CTCGTG) and BsoBI (manufactured by New England BioLabs: recognition sequence:CYCGRG).

According to the report published in a catalogue by New England BioLabs (2002-3 edition; p. 263) or; on its home page (http://www.neb.com/nebecomm/tech_reference/restriction_en zymes/fragment_size_by_cleavage.asp), the frequency of appearance of SalI in the mouse genome is once per 48 kb on average. Provided that the mouse genome has a 26.4 billion base pairs (National Center for Biotechnology Information U.S. National Library of Medicine as of; 1 Oct. 2004 http://www.ncbi.nlm.nih.gov/mapview/), the recognition sites for SalI will be about 55,000. And NotI and AscI have the frequency of appearance of once per 120 kb and 280 kb on average, respectively, they will have the recognition sites of about 22,000 and 9,400, respectively.

Accordingly, if SalI is used as the first restriction enzyme, about 55,000 DNA fragments may be obtained. However, provided that 70% of CG sequence is methylated in the genome (Non-Patent Document 1), SalI, the methylation-sensitive enzyme, cannot cleave about 38,500 sites, (0.70% of 55,000), and will produce only about 16,500 (30% of 55,000) DNA fragments, being expected to have 160 kb on average (26.4 billion divided by 16,500). Practically, it would be very difficult to handle such long DNA fragment.

The above fragment with % about 160 kb will be therefore treated and truncated with the second restriction enzyme Y to give a fragment with a handy length. The second restriction enzyme Y, unlike the first restriction enzyme X, is preferably a 4-base recognizing enzyme having a high frequency of appearance so as to obtain DNA fragment with a handy size. For this purpose, it will be desirable to use a methylation-insensitive enzyme as the second restriction enzyme Y. However, the use of an methylation-sensitive enzyme as the second restriction enzyme Y will of help in identifying a methylated region in a wide range. It is therefore possible to selectively use these enzymes depending on the subject of analysis.

Preferred examples of the second restriction enzyme Y include methylation-insensitive enzymes such as MspI (manufactured by Takara Bio. Inc.: recognition sequence:CCGG) and TaqI (manufactured by New England BioLabs: recognition sequence:TCGA), and methylation-sensitive enzymes such as HapII (manufactured by New England BioLabs: recognition sequence:CCGG) and HhaI (manufactured by New England BioLabs: recognition sequence:GCGC).

The DNA fragment population (DNA fragment library) thus prepared by cleavage with the first and second restriction enzymes shall therefore consist only of DNA fragments derived from the non-methylated regions.

Each DNA fragment member in the above DNA fragment library will be separated and detected depending on its chain length (molecular size). Specific methods for that are well known for those skilled in the art, for example, electrophoresis, liquid chromatography (HPLC) and time of flight mass spectrometer (TOF/MS) being generally used. For example, the separation and detection of the PCR products in electrophoresis may be carried out based on their migration length and peak.

Specifically, a gel electrophoresis using polyacrylamide gel usually handles a DNA fragment having a chain length of about 20 to 1,000 bases, and will show such an excellent separation efficiency as that of one base. However, if SalI and MspI (manufactured by Takara Bio. Inc.: recognition sequence:CCGG) are used as the first and second restriction enzymes, respectively, about 16,500 DNA fragments will be obtained by the first restriction enzyme SalI considering the effect by the 70% methylation. And about 33,000 DNA fragments having SalI and MspI at their both ends will then be obtained by the second restriction enzyme. In such case, it would be very difficult to sufficiently separate about 33,000 DNA fragments or more depending only on their chain length even if the electrophoresis could show the separation efficiency of one base.

The invention will therefore utilize High Coverage Expression Profiling (HiCEP) analysis disclosed in WO02/48352 pamphlet in order to separate such many kinds of the DNA fragments. According to this method, the DNA fragment library can be classified by 256 combinations of the two-base sequences flanking to the first and second restriction enzyme recognition sites of the DNA fragment cleaved by the first restriction enzyme X and the second restriction enzyme Y. As a result about 33,000 kinds of the DNA fragments will be classified into groups, each of which has about 129 kinds of the DNA fragments. These resulting 129 kinds of DNA fragments may be practically separated and quantified in electrophoresis. Furthermore, this method could sufficiently separate and identify the DNA fragments even if the CG methylation ratio is as low as 60% so that about 172 kinds of DNA fragments will be produced per each pair of the two-base sequences.

The adaptor is used to bind the primers to the DNA sequence in PCR. It may be optionally designed depending on the structure of the restriction enzymes and primers used in the reaction. The primers are usually 30-base long for performing a stable PCR.

The X primer, X1 primer, Y primer and Y1 primer have preferably 16 bases or more so as not to coincide with the subject RNA sequence wherever possible. Furthermore, it is necessary for these primers to satisfy the conditions generally required as a PCR primer, such as those described in “BioRad Experiment Illustrated (3) New Edition, Really Amplified PCR” Hiroki Nakayama, Shujunn Co., 2002, the second edition, the forth print. Each primer may be prepared in accordance with a general synthesizing method known for those skilled in the art (Letsinger et al., Nucleic Acids Research, 20, 1879-1882, 1992; Japanese Patent Application Publication Hei.11(1999)-08018).

It is further preferable to bind a labeling substance such as any fluorescent substance known for those skilled in the art to at least either end of the primers in order to ease the detection after PCR. For example, the suitable fluorescent substances include 6-carboxyfluorescein (FAM), 4,7,2′,4′,5′,7′-hexachloro-6-carboxyfluorescein (HEX), NED (Applied System Japan, Co.) and 6-carboxy-X-rhodamine (Rox).

The tag substance and a substance having a high affinity for the tag substance mean one of the substances that can specifically bind with each other with a high affinity. Any substances may be used for them as long as they specifically bind with each other with a high affinity. Unlimited examples of the combination of these substances include biotin and streptavidin, biotin and avidin, FITC and anti-FITC antibody, DIG and anti-DIG, protein A and mouse IgG, and latex particles, etc. The tag substance may be added to the DNA sequence under any suitable conditions known for those skilled in the art. The double-stranded DNA having the tag substance added thereto may be collected through a specific reaction between the tag substance and the substance having a high affinity for the tag substance.

Other conditions and apparatuses used in the HiCEP method may be referred to the description of WO02/48352 pamphlet. The resulting gene expression profile may be analyzed by means of any analyzing software known for those skilled in the art such as GeneScan (a trade mark: Applied BioSystems Japan, Co.)

It is preferable to carry out annealing of the X primer or X1 primer, and the Y primer or Y1 primer with the X adaptor and Y adaptor, respectively, at a temperature range of TmMAX+6° C.˜TmMAX+14° C. of the primer, so that the occurrence of false peaks due to mis-annealing of the primers can be diminished.

### EXAMPLES

Next, the invention will be described in detail with reference to an example corresponding to the first aspect of the present invention, however, it should be appreciated that the invention will not be limited in any way by the specific example.

DNA was extracted and purified from mouse ES cells and mouse thymus cells, and 5 μg each of the DNA was used.

(a) a step of cleaving a genomic DNA with a methylation-sensitive first restriction enzyme X recognizing CG sequence:

TABLE 1

| | | |
|---|---|---|
| Genomic DNA | 5 | μg |
| 10 × SalI buffer | 40 | μl |
| SalI (Takara Bio. Inc.) | 60 | U |

Brought to 400 μl with distilled water

The reaction was carried out at 37° C. for 3 hours. DNA was then concentrated and purified with ethanol precipitation. After being dried, the DNA was dissolved into 20 μl of TE solution.

(b) a step of binding an X adaptor comprising a complementary sequence to a cleaved site by the first restriction enzyme X, a complementary sequence to an X primer and a tag substance added to the other end of the complementary sequence to the cleaved site, to the cleaved site of the DNA fragment cleaved by the first restriction enzyme X in the step (a) so as to prepare a DNA fragment having the X adaptor bound to the cleaved site:

X adaptor

(SEQ ID No.1)

1. 5′-Biotin-AAGTATCGTCACGAGGCGTCCTACTGGC-3′

(SEQ ID No.2)

2. 5′-TCGAGCCAGTAGGACGCCTCGTGACGATACTT-3′

By annealing of the above two oligomers 1 and 2, the following X adaptor solution (100 μM) was prepared:

5′-Biotin-AAGTATCGTCACGAGGCGTCCTACTGGC-3′
3′ TTCATAGCAGTGCTCCGCAGGATGACCGAGCT-5′

TABLE 2

| | | |
|---|---|---|
| DNA cleaved by SalI and purified | 20 | μl |
| 100 μM × adaptor solution | 2 | μl |
| 10 × T4 DNA ligase buffer | 2.5 | μl |
| 10 mM ATP solution | 1 | μ1 |
| T4 DNA ligase (Takara Bio. Inc.) | 350 | U |

Brought to 27 μl with distilled water

The reaction was carried out at 16° C. for 6 hours. DNA was then concentrated and purified with ethanol precipitation. After being dried, the DNA was dissolved into 50 μl of TE solution.

(c) a step of cleaving the DNA fragment having the X adaptor bound thereto and prepared in the step (b) with a second restriction enzyme Y that does not cleave a sequence complementary to the X primer:

TABLE 3

| | | |
|---|---|---|
| Solution of DNA bound with X adaptor | 50 | μl |
| 10 × MspI buffer | 10 | μl |
| 0.1% BSA | 10 | μl |
| MspI (Takara Bio. Inc.) | 60 | U |

Brought to 100 μl with distilled water and reacted at 37° C. for 3 hours.

(d) a step of separating and purifying the DNA fragment having the X adaptor bound thereto and cleaved in the step (c) with a substance having a high affinity for the tag substance added to the X adaptor:

To a solution of the DNA fragments cleaved with MapI was added a solution of Streptavidine-coated magnetic beads (DYNAL Co.) so that the DNA fragments having the X adaptor with Biotin-tag were adsorbed. The magnetic beads were collected by using magnet, and supernatant was discarded. To this was added 500 μl of 1×B/W (washing solution for the magnetic beads) and suspended. Again, the magnetic beads were collected by using magnet, and supernatant was discarded. The resulting magnetic beads were suspended into 20 μl of distilled water.

(e) a step of binding a Y adaptor comprising a complementary sequence to the cleaved site by the second restriction enzyme Y and a complementary sequence to a Y primer, to the cleaved site by the second restriction enzyme Y of the DNA fragment having the X adaptor bound thereto and purified in the step (d) so as to prepare a DNA fragment having the X adaptor and Y adaptor bound to its both ends:

Y adaptor

(Seq ID No.3)

3. 5′-AATGGCTACACGAACTCGGTTCATGACA-3′

(Seq ID No.4)

4. 5′-CGTGTCATGAACCGAGTTCGTGTAGCCATT-3′

By annealing of the above two oligomers 3 and 4, the following Y adaptor solution (100 μm) was prepared:

5′-AATGGCTACACGAACTCGGTTCATGACA-3′
3′-TTACCGATGTGCTTGAGCCAAGTACTGTGC-5′

TABLE 4

| | | |
|---|---|---|
| magnetic bead suspension | 20 | μl |
| 100 μM Y adaptor solution | 2 | μl |
| 10 × T4 DNA ligase buffer | 2.5 | μl |
| 10 mM ATP solution | 1 | μl |
| T4 DNA ligase (Takara Bio. Inc.) | 350 | U |

Brought to 27 μl with distilled water

The reaction was carried out at 25° C. for 6 hours. The magnetic beads were collected and the supernatant was discarded. To this was added 500 μl of 1×B/W (washing solution for the magnetic beads) and suspended. Again, the magnetic beads were collected by using magnet, and supernatant was discarded. The resulting magnetic beads were suspended into 40 μl of distilled water.

(f) a step of amplifying the DNA fragment having the X adaptor and Y adaptor bound to its both ends and prepared in the step (e) by means of PCR with the use of said DNA fragment as a template and a primer set of the X primer and Y primer:

TABLE 5

| | |
|---|---|
| Solution obtained in the step (e) | 10 μl |
| 10 × PCR Buffer | 5 μl |
| 25 mM MgCl₂ | 5 μl |
| dNTP Mixture (2.5 mM each) | 8 μl |
| Taq Polymerase (5 u/μl) | 1 μl |
| X Primer (100 pmol/μl) | 0.5 μl |
| Y Primer (100 pmol/μl) | 0.5 μl |

Brought to 50 μl with distilled water and set into a PCR apparatus.

TABLE 6
PCT temperature steps:

| | |
|---|---|
| Step 1: | 95° C. for 5 min |
| Step 2: | (95° C. for 20 sec, 68° C. for 15 min) × seven times |
| Step 3: | 60° C. for 30 min |

The PCR solution was purified with a PCR product purification kit, and unreacted X primer and Y primer were removed. The resulting DNA solution was dissolved into 40 μl of distilled water.

(g) a step of PCR with the use of a double-stranded DNA sequence prepared in the step (e) as a template and a primer set of X1 primer comprising a two-base sequence of N₁N₂ (N₁ and N₂ are a base selected from the group consisting of adenine, thymine, guanine and cytosine, being the same or different with each other) at its 3′ end and Y1 primer comprising a two-base sequence of N₃N₄ (N₃ and N₄ are a base selected from the group consisting of adenine, thymine, guanine and cytosine, being the same or different with each other) at its 3′ end:

TABLE 7

| | |
|---|---|
| X1 primer: | Oligomer having the sequence |
| | complementary to the X adaptor and the |
| | two-base sequence combination, and |
| | labeled with a fluorescent substance at |
| | its 5′ end |
| FAM: | fluorescein label |
| HEX: | 5′-Hexafluorocerin label |
| NED: | fluorescent pigment label manufactured |
| | by Applied Biosystems |
| X-AA | FAM-GCGTCCTACTGGCTCGACAA |
| X-AC | FAM-GCGTCCTACTGGCTCGACAC |
| X-AG | FAM-GCGTCCTACTGGCTCGACAG |
| X-AT | FAM-GCGTCCTACTGGCTCGACAT |
| X-CA | NED-GCGTCCTACTGGCTCGACCA |
| X-CC | NED-GCGTCCTACTGGCTCGACCC |
| X-CG | NED-GCGTCCTACTGGCTCGACCG |
| X-CT | HEX-GCGTCCTACTGGCTCGACCT |
| X-GA | NED-GCGTCCTACTGGCTCGACGA |
| X-GC | HEX-GCGTCCTACTGGCTCGACGC |
| X-GG | HEX-GCGTCCTACTGGCTCGACGG |
| X-GT | FAM-GCGTCCTACTGGCTCGACGT |
| X-TA | NED-GCGTCCTACTGGCTCGACTA |
| X-TC | HEX-GCGTCCTACTGGCTCGACTC |
| X-TG | HEX-GCGTCCTACTGGCTCGACTG |
| X-TT | FAM-GCGTCCTACTGGCTCGACTT |

TABLE 8

| | |
|---|---|
| Y1 primer: | Oligomer having the sequence |
| | complementary to the y adaptor and the |
| | two-base sequence combination: |
| Y-AA | ACTCGGTTCATGACACGGAA |
| Y-AC | ACTCGGTTCATGACACGGAC |
| Y-AG | ACTCGGTTCATGACACGGAG |
| Y-AT | ACTCGGTTCATGACACGGAT |
| Y-CA | ACTCGGTTCATGACACGGCA |
| Y-CC | ACTCGGTTCATGACACGGCC |
| Y-CG | ACTCGGTTCATGACACGGCG |
| Y-CT | ACTCGGTTCATGACACGGCT |
| Y-GA | ACTCGGTTCATGACACGGGA |
| Y-GC | ACTCGGTTCATGACACGGGC |
| Y-GG | ACTCGGTTCATGACACGGGG |
| Y-GT | ACTCGGTTCATGACACGGGT |
| Y-TA | ACTCGGTTCATGACACGGTA |
| Y-TC | ACTCGGTTCATGACACGGTC |
| Y-TG | ACTCGGTTCATGACACGGTG |
| Y-TT | ACTCGGTTCATGACACGGTT |

The above 32 kinds of the oligomers were synthesized, and solution comprising each of the above 32 oligomers (2 μM) was prepared. Each combination of X1 primer and Y1 primer (2 μl each) was prepared in line accordance with the following table and divided into 256 PCR tubes, respectively.

TABLE 9
Combination of X1 primers and Y1 primers

| X1-Y1 | X1-Y1 | X1-Y1 | X1-Y1 |
|---|---|---|---|
| AA-AA | AC-AA | AG-AA | AT-AA |
| AA-AC | AC-AC | AG-AC | AT-AC |
| AA-AG | AC-AG | AG-AG | AT-AG |
| AA-AT | AC-AT | AG-AT | AT-AT |
| AA-CA | AC-CA | AG-CA | AT-CA |
| AA-CC | AC-CC | AG-CC | AT-CC |
| AA-CG | AC-CG | AG-CG | AT-CG |
| AA-CT | AC-CT | AG-CT | AT-CT |
| AA-GA | AC-GA | AG-GA | AT-GA |
| AA-GC | AC-GC | AG-GC | AT-GC |
| AA-GG | AC-GG | AG-GG | AT-GG |
| AA-GT | AC-GT | AG-GT | AT-GT |
| AA-TA | AC-TA | AG-TA | AT-TA |
| AA-TC | AC-TC | AG-TC | AT-TC |
| AA-TG | AC-TG | AG-TG | AT-TG |
| AA-TT | AC-TT | AG-TT | AT-TT |
| TG-AA | CA-AA | GA-AA | TA-AA |
| TG-AC | CA-AC | GA-AC | TA-AC |
| TG-AG | CA-AG | GA-AG | TA-AG |
| TG-AT | CA-AT | GA-AT | TA-AT |
| TG-CA | CA-CA | GA-CA | TA-CA |
| TG-CC | CA-CC | GA-CC | TA-CC |
| TG-CG | CA-CG | GA-CG | TA-CG |
| TG-CT | CA-CT | GA-CT | TA-CT |
| TG-GA | CA-GA | GA-GA | TA-GA |
| TG-GC | CA-GC | GA-GC | TA-GC |
| TG-GG | CA-GG | GA-GG | TA-GG |
| TG-GT | CA-GT | GA-GT | TA-GT |
| TG-TA | CA-TA | GA-TA | TA-TA |
| TG-TC | CA-TC | GA-TC | TA-TC |
| TG-TG | CA-TG | GA-TG | TA-TG |
| TG-TT | CA-TT | GA-TT | TA-TT |
| X1-Y1 | X1-Y1 | X1-Y1 | X1-Y1 |
| TT-AA | GT-AA | CT-AA | TC-AA |
| TT-AC | GT-AC | CT-AC | TC-AC |
| TT-AG | GT-AG | CT-AG | TC-AG |
| TT-AT | GT-AT | CT-AT | TC-AT |
| TT-CA | GT-CA | CT-CA | TC-CA |
| TT-CC | GT-CC | CT-CC | TC-CC |
| TT-CG | GT-CG | CT-CG | TC-CG |
| TT-CT | GT-CT | CT-CT | TC-CT |
| TT-GA | GT-GA | CT-GA | TC-GA |
| TT-GC | GT-GC | CT-GC | TC-GC |
| TT-GG | GT-GG | CT-GG | TC-GG |
| TT-GT | GT-GT | CT-GT | TC-GT |
| TT-TA | GT-TA | CT-TA | TC-TA |
| TT-TC | GT-TC | CT-TC | TC-TC |
| TT-TG | GT-TG | CT-TG | TC-TG |
| TT-TT | GT-TT | CT-TT | TC-TT |
| CC-AA | CG-AA | GC-AA | GG-AA |
| CC-AC | CG-AC | GC-AC | GG-AC |
| CC-AG | CG-AG | GC-AG | GG-AG |
| CC-AT | CG-AT | GC-AT | GG-AT |
| CC-CA | CG-CA | GC-CA | GG-CA |
| CC-CC | CG-CC | GC-CC | GG-CC |
| CC-CG | CG-CG | GC-CG | GG-CG |
| CC-CT | CG-CT | GC-CT | GG-CT |
| CC-GA | CG-GA | GC-GA | GG-GA |
| CC-GC | CG-GC | GC-GC | GG-GC |
| CC-GG | CG-GG | GC-GG | GG-GG |
| CC-GT | CG-GT | GC-GT | GG-GT |
| CC-TA | CG-TA | GC-TA | GG-TA |
| CC-TC | CG-TC | GC-TC | GG-TC |
| CC-TG | CG-TG | GC-TG | GG-TG |
| CC-TT | CG-TT | GC-TT | GG-TT |

TABLE 10
PCR reaction solution

| | | |
|---|---|---|
| Solution obtained in the step (g) | 5 | μl |
| Water | 2,515 | μl |
| 10 × PCR Buffer | 600 | μl |
| 25 mM MgCl₂ | 600 | μl |
| dNTP Mixture (2.5 mM each) | 960 | μl |
| Taq Polymerase (5 u/μl) | 120 | μl |
| Total | 4,800 | μl |

PCR reaction solution was divided into each tube (16 μl) and set in the apparatus for PCR.

PCT Temperature steps:

| | |
|---|---|
| Step 1: | 95° C., 1 min; |
| Step 2: | (98° C., 20 sec; 71.5° C., 30 sec, 72° C., 1 min) × 28 times; |
| Step 3: | 60° C., 30 min. |

(h) a step of separating and detecting the resulting PCR products in accordance with their chain length:

The PCR product prepared in the step (g) was subjected to electrophoresis and analysis using ABI PRISM (trade mark) 3100 Genetic Analyzer (Applied Biosystems Co.) in accordance with its manual. The analysis of the 256 tubes of each lot of the samples revealed that electrophoresis patterns obtained in all of the samples were different with each other for the same combination of the X1 primer and Y1 primer.

### INDUSTRIAL APPLICABILITY

According to the present invention, a number of the non-methylated regions in the genome of two or more types of cells can be simultaneously detected, and exhaustively compared and analyzed.

1. A method for the detection of a non-methylated region in genome comprising:
(a) a step of cleaving a genomic DNA with a first restriction enzyme X;
(b) a step of binding an X adaptor comprising a complementary sequence to a cleaved site by the first restriction enzyme X, a complementary sequence to an X primer and a tag substance added to the other end of the complementary sequence to the cleaved site, to the cleaved site of the DNA fragment cleaved by the first restriction enzyme X in the step (a) so as to prepare a DNA fragment having the X adaptor bound to the cleaved site;
(c) a step of cleaving the DNA fragment having the X adaptor bound thereto and prepared in the step (b) with a second restriction enzyme. Y that does not cleave a sequence complementary to the X primer;
(d) a step of separating and purifying the DNA fragment having the X adaptor bound thereto and cleaved in the step (c) with a substance having a high affinity for the tag substance added to the X adaptor;
(e) a step of binding a Y adaptor comprising a complementary sequence to a cleaved site by the second restriction enzyme Y and a complementary sequence to a Y primer, to the cleaved site by the second restriction enzyme Y of the DNA fragment having the X adaptor bound thereto and purified in the step (d) so as to prepare a DNA fragment having the X adaptor and Y adaptor bound to its both ends;
(g) a step of PCR with the use of a double-stranded DNA sequence prepared in the step (e) as a template and a primer set of X1 primer comprising a two-base sequence of N₁N₂ (N₁ and N₂ are a base selected from the group consisting of adenine, thymine, guanine and cytosine, being the same or different with each other) at its 3′ end and Y1 primer comprising a two-base sequence of N₃N₄ (N₃ and N₄ are a base selected from the group consisting of adenine, thymine, guanine and cytosine, being the same or different with each other) at its 3′ end; and
(h) a step of separating and detecting the resulting PCR products in accordance with their chain length.
wherein either of the first restriction enzyme X and the second restriction enzyme Y is a methylation-sensitive enzyme.

2. A method according to claim 1 further comprising the following step (f) between the steps (e) and (g):
(f) a step of amplifying the DNA fragment having the X adaptor and Y adaptor bound to its both ends and prepared in the step (e) by means of PCR with the use of said DNA fragment as a template and a primer set of the X primer and Y primer.

3. A method according to claim 2 wherein the number of PCR cycles is set between 7 and 10 so as to amplify the DNA fragment 128-1024 times.

4. The method according to claim 1, further comprising (i) a step of identifying the detected peak.

5. The method according to claim 1 wherein the first restriction enzyme X is a methylation-sensitive enzyme.

6. A method according to claim 5 wherein the first restriction enzyme X is a methylation-sensitive enzyme that recognizes 6 or 8 bases.

7. The method according to claim 1 wherein the first restriction enzyme X is a methylation-insensitive enzyme and the second restriction enzyme Y is a methylation-sensitive enzyme.

8. The method according to claim 1 wherein the first restriction enzyme X is a methylation-sensitive enzyme SalI and the second restriction enzyme Y is a methylation-insensitive enzyme MspI.

9. The method according to claim 1 wherein the separation and detection of the PCR products in accordance with their chain length is carried out based on their migration length and peak.

10. A method for analyzing the change in a transcriptionally-active region in a genome comprising detecting a non-methylated region in the genome, which is derived from more than two kinds of cells, by the method according to claim 1, and comparing the results to analyze a difference in the non-methylated region.


SEQUENCE LISTING (38 sequences)

SEQ ID NO: 1 - DNA, 28 bp, Artificial Sequence
  Synthetic oligonucleotide X-Adaptor
aagtatcgtcacgaggcgtcctactggc

SEQ ID NO: 2 - DNA, 32 bp, Artificial Sequence
  Synthetic oligonucleotide X-Adaptor
tcgagccagtaggacgcctcgtgacgatactt

SEQ ID NO: 3 - DNA, 28 bp, Artificial Sequence
  Synthetic oligonucleotide Y-Adaptor
aatggctacacgaactcggttcatgaca

SEQ ID NO: 4 - DNA, 30 bp, Artificial Sequence
  Synthetic oligonucleotide Y-Adaptor
cgtgtcatgaaccgagttcgtgtagccatt

SEQ ID NO: 5 - DNA, 32 bp, Artificial Sequence
  Synthetic oligonucleotide X-Primer
aagtatcgtcacgaggcgtcctactggctcga

SEQ ID NO: 6 - DNA, 31 bp, Artificial Sequence
  Synthetic oligonucleotide Y-Primer
aatggctacacgaactcggttcatgacacgg

SEQ ID NO: 7 - DNA, 20 bp, Artificial Sequence
  Synthetic oligonucleotide
gcgtcctactggctcgacaa

SEQ ID NO: 8 - DNA, 20 bp, Artificial Sequence
  Synthetic oligonucleotide
gcgtcctactggctcgacac

SEQ ID NO: 9 - DNA, 20 bp, Artificial Sequence
  Synthetic oligonucleotide
gcgtcctactggctcgacag

SEQ ID NO: 10 - DNA, 20 bp, Artificial Sequence
  Synthetic oligonucleotide
gcgtcctactggctcgacat

SEQ ID NO: 11 - DNA, 20 bp, Artificial Sequence
  Synthetic oligonucleotide
gcgtcctactggctcgacca

SEQ ID NO: 12 - DNA, 20 bp, Artificial Sequence
  Synthetic oligonucleotide
gcgtcctactggctcgaccc

SEQ ID NO: 13 - DNA, 20 bp, Artificial Sequence
  Synthetic oligonucleotide
gcgtcctactggctcgaccg

SEQ ID NO: 14 - DNA, 20 bp, Artificial Sequence
  Synthetic oligonucleotide
gcgtcctactggctcgacct

SEQ ID NO: 15 - DNA, 20 bp, Artificial Sequence
  Synthetic oligonucleotide
gcgtcctactggctcgacga

SEQ ID NO: 16 - DNA, 20 bp, Artificial Sequence
  Synthetic oligonucleotide
gcgtcctactggctcgacgc

SEQ ID NO: 17 - DNA, 20 bp, Artificial Sequence
  Synthetic oligonucleotide
gcgtcctactggctcgacgg

SEQ ID NO: 18 - DNA, 20 bp, Artificial Sequence
  Synthetic oligonucleotide
gcgtcctactggctcgacgt

SEQ ID NO: 19 - DNA, 20 bp, Artificial Sequence
  Synthetic oligonucleotide
gcgtcctactggctcgacta

SEQ ID NO: 20 - DNA, 20 bp, Artificial Sequence
  Synthetic oligonucleotide
gcgtcctactggctcgactc

SEQ ID NO: 21 - DNA, 20 bp, Artificial Sequence
  Synthetic oligonucleotide
gcgtcctactggctcgactg

SEQ ID NO: 22 - DNA, 20 bp, Artificial Sequence
  Synthetic oligonucleotide
gcgtcctactggctcgactt

SEQ ID NO: 23 - DNA, 20 bp, Artificial Sequence
  Synthetic oligonucleotide
actcggttcatgacacggaa

SEQ ID NO: 24 - DNA, 20 bp, Artificial Sequence
  Synthetic oligonucleotide
actcggttcatgacacggac

SEQ ID NO: 25 - DNA, 20 bp, Artificial Sequence
  Synthetic oligonucleotide
actcggttcatgacacggag

SEQ ID NO: 26 - DNA, 20 bp, Artificial Sequence
  Synthetic oligonucleotide
actcggttcatgacacggat

SEQ ID NO: 27 - DNA, 20 bp, Artificial Sequence
  Synthetic oligonucleotide
actcggttcatgacacggca

SEQ ID NO: 28 - DNA, 20 bp, Artificial Sequence
  Synthetic oligonucleotide
actcggttcatgacacggcc

SEQ ID NO: 29 - DNA, 20 bp, Artificial Sequence
  Synthetic oligonucleotide
actcggttcatgacacggcg

SEQ ID NO: 30 - DNA, 20 bp, Artificial Sequence
  Synthetic oligonucleotide
actcggttcatgacacggct

SEQ ID NO: 31 - DNA, 20 bp, Artificial Sequence
  Synthetic oligonucleotide
actcggttcatgacacggga

SEQ ID NO: 32 - DNA, 20 bp, Artificial Sequence
  Synthetic oligonucleotide
actcggttcatgacacgggc

SEQ ID NO: 33 - DNA, 20 bp, Artificial Sequence
  Synthetic oligonucleotide
actcggttcatgacacgggg

SEQ ID NO: 34 - DNA, 20 bp, Artificial Sequence
  Synthetic oligonucleotide
actcggttcatgacacgggt

SEQ ID NO: 35 - DNA, 20 bp, Artificial Sequence
  Synthetic oligonucleotide
actcggttcatgacacggta

SEQ ID NO: 36 - DNA, 20 bp, Artificial Sequence
  Synthetic oligonucleotide
actcggttcatgacacggtc

SEQ ID NO: 37 - DNA, 20 bp, Artificial Sequence
  Synthetic oligonucleotide
actcggttcatgacacggtg

SEQ ID NO: 38 - DNA, 20 bp, Artificial Sequence
  Synthetic oligonucleotide
actcggttcatgacacggtt